(19) United States
(12) Patent Application Publication
(10) Pub. No.: US 2022/0339402 A1
(43) Pub. Date: Oct. 27, 2022

(54) Tunneled Intravascular Catheters, Catheter Systems, and Related Methods

(71) Applicant: Ohio State Innovation Foundation, Columbus, OH (US)

(72) Inventors: Mark Ruegsegger, Columbus, OH (US); Khaled Boubes, Columbus, OH (US); Scott Kelly, Columbus, OH (US); Ashita Singhal, Columbus, OH (US); Kayla Hepler, Columbus, OH (US); Cameron Bezold, Columbus, OH (US); Hannah Gruensfelder, Columbus, OH (US); Christopher Hummel, Columbus, OH (US); Sharon Brickey, Columbus, OH (US); Jad Hussein, Columbus, OH (US); Ryan McIlvaine, Columbus, OH (US); Margaret Veres, Columbus, OH (US)

(21) Appl. No.: 17/728,670

(22) Filed: Apr. 25, 2022

Related U.S. Application Data
(60) Provisional application No. 63/178,921, filed on Apr. 23, 2021.
(60) Provisional application No. 63/324,630, filed on Mar. 28, 2022.

(51) Int. Cl. A61M 25/00; A61F 2/90; A61F 2/92; A61F 2/00

(52) U.S. Cl. CPC A61M 25/0029; A61F 2/90; A61F 2/92; A61F 2/0077; A61M 2025/0006; A61F 2002/0086

(57) ABSTRACT

A catheter may include a catheter body having a tubular shape, a cuff encircling the catheter body, and a wire mesh attached to and encircling the catheter body. The cuff may be configured for facilitating fibrous tissue growth. The wire mesh may be configured for radially expanding away from the catheter body.

### CROSS-REFERENCE TO RELATED APPLICATIONS

This application claims the benefit of, and priority to, U.S. Provisional Patent Application No. 63/178,921, filed on Apr. 23, 2021, and titled “Intravascular Catheter Cuff,” and U.S. Provisional Patent Application No. 63/324,630, filed on Mar. 28, 2022, and titled “Tunneled Central Venous Catheter Removal Device,” the disclosures of which are expressly incorporated herein by reference in their entirety.

### FIELD OF THE DISCLOSURE

The present disclosure relates generally to catheters and more particularly to devices, systems, and methods for removal of a tunneled intravascular catheter having a cuff that facilitates fibrous tissue growth when the catheter is implanted in a patient.

### BACKGROUND OF THE DISCLOSURE

Millions of patients each year require long-term, secure intravenous access for a broad range of conditions that require fluid administration, including cancer, hemodynamic instability, and kidney failure. Tunneled catheters meet this demand due to their ability to induce formation of a fibrin sheath around a cuff of the catheter, which anchors the catheter in place and creates a barrier to the external environment. Tunneled intravascular catheters transition from tubing to a vein using a polyethylene terephthalate (PET) cuff that is placed subcutaneously. To anchor the catheter, the Dacron (PET) cuff induces fibrosis. The fibrin sheath provides secure attachment and protection from infection. The tunneled intravascular catheter is a highly common medical device used for treatments requiring medium to long-term central vascular access, including administration of hemodialysis when placement of an AV fistula is not possible, chemotherapy, total parenteral nutrition, blood transfusions, and plasmapheresis. This category of catheter is placed through an incision in the chest and is “tunneled” under the skin until it enters a central vein (commonly, the internal jugular vein). FIG. 1 illustrates placement of an example tunneled venous catheter implanted in a patient. As shown, the catheter includes a knitted or woven Dacron cuff that sits within the tunneled pocket. Within a few weeks of implantation, the Dacron cuff will facilitate fibrous tissue growth which secures the catheter in place and creates a biological seal from the environment to prevent infection. While this fibrin growth is highly beneficial during catheter implantation, it complicates the extraction process, especially when the catheter has been in place for a long period and tissue ingrowth is robust.

The current removal procedure requires a surgeon to insert a blunt dissection tool, typically a hemostat, into the exit site and scrape away the adhered fibrous tissue from the cuff and the surrounding area until the catheter can be removed. This procedure generally increases the risk of complications, such as the disruption of a healed exit site wound caused by the insertion of the blunt instrument and the subsequent rigorous movement required to dissect tissue. In addition to complications, this extraction typically requires a surgeon trained in the specific removal procedure as the fibrous tissue may be difficult to access and detach. Also, this procedure may introduce significant operating room costs, as the tedious tissue removal process can demand more than 30 minutes of a surgeon's time. Tunneled intravascular catheter removal time varies from patient to patient, and often increases as time in the body increases. Modifications to the tunneled intravascular catheter that increase removal efficiency and safety could reduce the amount of trauma caused by the procedure as well as minimize procedure time and cost. Additionally, a more efficient removal procedure could increase the number of medical professionals qualified to perform the process, as catheter extraction would require less skill to complete correctly.

A need therefore exists for improved devices, systems, and methods for removal of a tunneled catheter having a cuff that facilitates fibrous tissue growth, which devices, systems, and methods may overcome one or more of the challenges associated with current techniques.

### SUMMARY OF THE DISCLOSURE

The present disclosure provides catheters, catheter systems, and related methods of using such catheters and catheter systems.

In one aspect, a catheter is provided. In one embodiment, the catheter may include a catheter body having a tubular shape, a cuff encircling the catheter body, and a wire mesh attached to and encircling the catheter body. The cuff may be configured for facilitating fibrous tissue growth. The wire mesh may be configured for radially expanding away from the catheter body.

In some embodiments, the catheter may be a tunneled catheter. In some embodiments, the cuff may be formed of a knitted or woven material. In some embodiments, the cuff may be formed of polyethylene terephthalate. In some embodiments, the wire mesh may have a stent configuration. In some embodiments, the wire mesh may extend over the cuff and be configured for radially expanding away from the cuff. In some embodiments, the cuff may be fixedly attached to the catheter body. In some embodiments, the wire mesh may extend between the catheter body and the cuff and be configured for breaking the cuff upon radial expansion of the wire. In some embodiments, the cuff may be removably attached to the wire mesh. In some embodiments, the wire mesh may have a distal end and a proximal end, and the distal end of the wire mesh may be fixedly attached to the catheter body. In some embodiments, the proximal end of the wire mesh may be removably attached to the catheter body. In some embodiments, the catheter also may include a collar fixedly attached to the wire mesh and encircling the catheter body. In some embodiments, the collar may be removably attached to the catheter body.

In another aspect, a catheter system is provided. In one embodiment, the catheter system may include a catheter and a tool. The catheter may include a catheter body having a tubular shape, a cuff attached to and encircling the catheter body, a wire mesh attached to and encircling the catheter body, and a collar attached to the wire mesh and encircling the catheter body. The cuff may be configured for facilitating fibrous tissue growth. The wire mesh may extend over the cuff and be configured for radially expanding away from the cuff. The tool may be configured for positioning over a portion of the catheter body and for removably coupling to the collar to facilitate radial expansion of the wire mesh.

In some embodiments, the tool may be configured for removably coupling to the collar via a twist-lock mechanism. In some embodiments, the collar may include a plurality of protrusions, and the tool may include a plurality of slots configured for receiving the protrusions to removably couple the tool to the collar.

In still another aspect, a method for removing a catheter from a patient is provided. The catheter may include a catheter body, a cuff with fibrous tissue growth, and a wire mesh encircling the catheter body. In one embodiment, the method may include radially expanding the wire mesh away from the catheter body, and pulling at least the catheter body and the wire mesh out of the patient.

In some embodiments, the wire mesh may extend over the cuff, radially expanding the wire mesh away from the catheter body may include radially expanding the wire mesh away from the cuff and separating the fibrous tissue growth from the cuff, and pulling at least the catheter body and the wire mesh out of the patient may include pulling the catheter body, the wire mesh, and the cuff out of the patient. In some embodiments, the wire mesh may extend between the catheter body and the cuff, and radially expanding the wire mesh away from the catheter body may include breaking the cuff and separating the wire mesh from the cuff.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1 is a schematic diagram illustrating placement of an example tunneled venous catheter implanted in a patient.

FIG. 2A is a perspective view of an example catheter in accordance with embodiments of the disclosure, showing a catheter body, a cuff, a wire mesh, a collar, and a sleeve of the catheter.

FIG. 2B is a detailed perspective view of a portion of the catheter of FIG. 2A, showing a portion of the catheter body, the cuff, the wire mesh, the collar, and the sleeve of the catheter.

FIG. 2C is a detailed perspective view of a portion of the catheter of FIG. 2A and an example tool as may be used with the catheter as part of a catheter system, showing the tool in an open configuration, and showing a portion of the catheter body, the wire mesh, and the collar of the catheter.

FIG. 2D is a detailed side view of a portion of the catheter of FIG. 2A and a portion of the tool, showing the tool in a closed configuration, protrusions of the collar received within slots of the tool, and the wire mesh in a radially collapsed configuration.

FIG. 2E is a detailed side view of a portion of the catheter of FIG. 2A and a portion of the tool, showing the tool in the closed configuration, the protrusions of the collar received within the slots of the tool, and the wire mesh in a radially expanded configuration.

FIG. 3 is a perspective view of an example catheter and an example tool as may be used with the catheter as part of a catheter system in accordance with embodiments of the disclosure, showing a catheter body, a cuff, a wire mesh, a collar, and a sleeve of the catheter.

FIG. 4A is a perspective view of an example catheter in accordance with embodiments of the disclosure, showing a catheter body, a cuff, a wire mesh, a temporary covering, and a sleeve of the catheter, with the wire mesh in a radially collapsed configuration.

FIG. 4B is a perspective view of a portion of the catheter of FIG. 4A, showing the catheter body, the cuff, the wire mesh, and the sleeve of the catheter, with the temporary covering removed, the wire mesh in a radially expanded configuration, and the cuff broken by radial expansion of the wire mesh.

FIG. 5 is a perspective view of an example catheter and an example tool as may be used with the catheter as part of a catheter system in accordance with embodiments of the disclosure, showing a catheter body, a cuff, a wire mesh, a collar, and a sleeve of the catheter.

FIG. 6 is a perspective view of an example catheter and an example inflation device as may be used with the catheter as part of a catheter system in accordance with embodiments of the disclosure, showing a catheter body, a cuff, and a tube of the catheter.

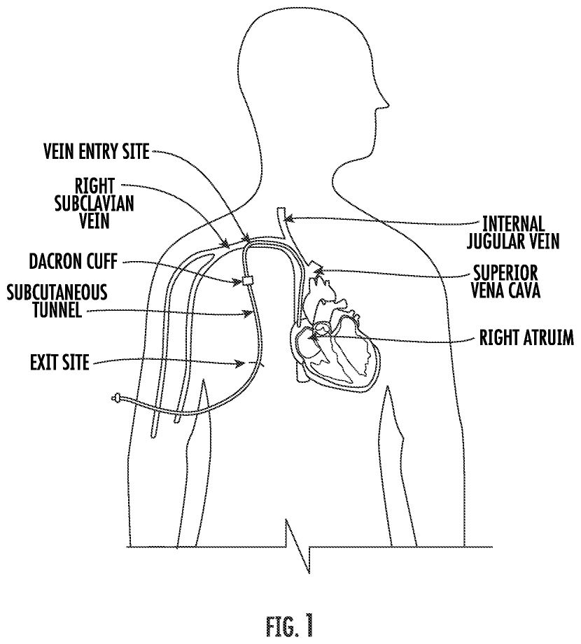

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

### Overview

The present disclosure provides embodiments of catheters, catheter systems, and related methods of their use, which may overcome one or more of the challenges associated with removal of conventional tunneled catheters, as discussed above. As described herein, a tunneled intravascular catheter may be provided with features to facilitate removal of the catheter from a patient after being implanted for a period of time. Similar to conventional tunneled catheters, the catheter may include a cuff that induces fibrous tissue growth, presenting challenges for removal of the catheter from the patient. In some embodiments, the catheter may include an expandable wire mesh that eases catheter removal. In some embodiments, expansion of the wire mesh may cause the fibrous tissue to separate from the cuff, thereby allowing the catheter to be more easily removed from the patient. In other embodiments, expansion of the wire mesh may cause the cuff to break and separate from a remainder of the catheter, thereby allowing the remainder of the catheter to be easily removed from the patient. In such embodiments, the cuff may be formed of a biodegradable material, such that the cuff may safely degrade within the patient. In some embodiments, a separate tool may be used with the catheter to streamline the catheter extraction process. In particular, the tool may be removably coupled to the catheter and used to facilitate expansion of the wire mesh. In some embodiments, the catheter may include an expandable balloon instead of an expandable wire mesh. In such embodiments, expansion of the balloon may cause the fibrous tissue to separate from the cuff, thereby allowing the catheter to be more easily removed from the patient. In some embodiments, the catheters described herein may be particularly useful as central tunneled catheters for hemodialysis. However, the same or similar configurations of the catheters also may be useful for other treatments, such as chemotherapy, and other cuffed (but non-central) catheter types, like peritoneal dialysis catheters.

Still other benefits and advantages of the catheters, catheter systems, and related methods provided herein over existing technology will be appreciated by those of ordinary skill in the art from the following description and the appended drawings.

### Example Catheters, Catheter Systems, and Methods

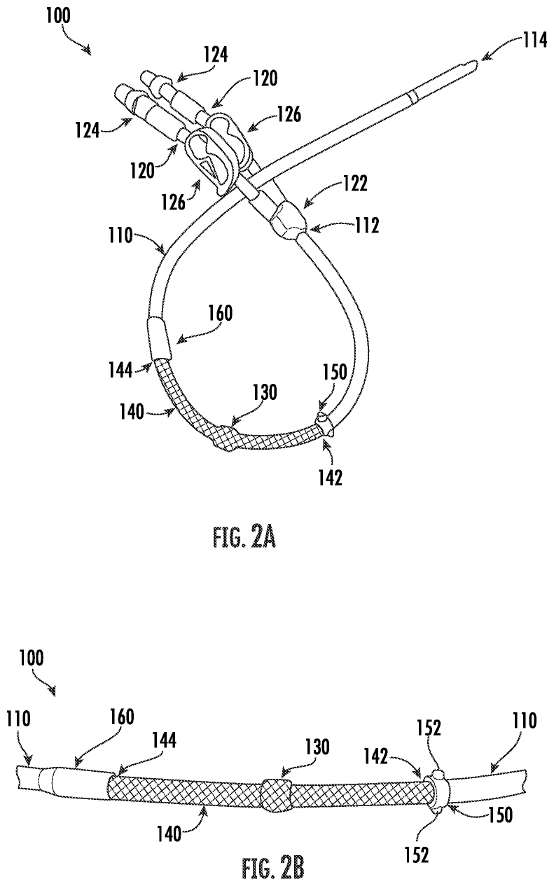

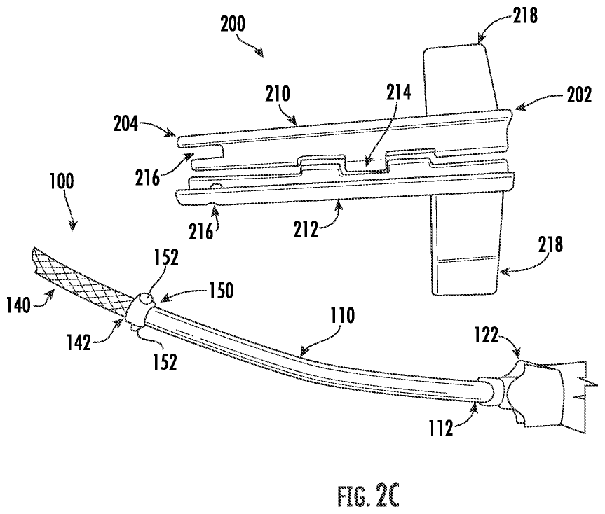

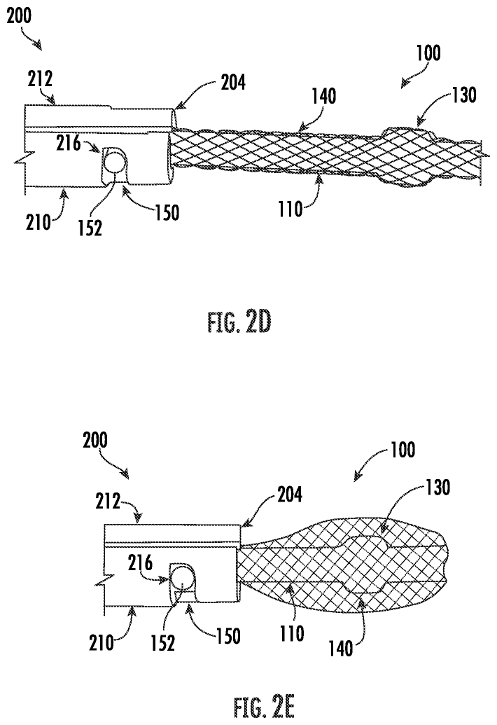

Referring now to the drawings, FIGS. 2A-2E illustrate an example catheter 100 (which also may be referred to as a “tunneled catheter” or a “tunneled intravascular catheter”) and an example tool 200 in accordance with embodiments of the disclosure. The catheter 100 and the tool 200 together may form a catheter system. As shown, the catheter may include a catheter body 110, a pair of branches 120, a cuff 130, a wire mesh 140, a collar 150, and a sleeve 160. As described above, the cuff 130 may be configured for facilitating fibrous tissue growth upon implantation of the catheter 100 for a period of time, and the fibrous tissue may complicate removal of the catheter 100 from the patient. However, as described herein, the wire mesh 140 may be used to facilitate separation of the fibrous tissue growth from the cuff 130 when removal is desired.

The catheter body 110 may have an elongate, tubular shape, with a proximal end 112 and a distal end 114. The catheter body 110 may include one or more lumens extending from the proximal end 112 to the distal end 114 for delivering fluid into and/or withdrawing fluid from a patient. The branches 120 may be coupled to the catheter body 110 via a coupler 122. Each of the branches 120 may have an elongate, tubular shape including one or more lumens in fluid communication with one or more of the lumens of the catheter body 110. In some embodiments, as shown, each of the branches 120 may have a port 124 attached thereto and configured for connecting to a fluid source or a drainage reservoir. In some embodiments, as shown, each of the branches 120 may have a clamp 126 positioned thereon and configured for selectively controlling fluid flow through the branch 120, when desired.

The cuff 130 may be attached to the catheter body 110 and may encircle the catheter body 110, similar to a conventional cuff In some embodiments, the cuff 130 may be fixedly attached to the catheter body 110. The cuff 130 may be configured for facilitating fibrous tissue growth. In some embodiments, the cuff 130 may be formed of a knitted or woven material to facilitate tissue ingrowth. In some embodiments, the cuff 130 may be formed of PTE.

As shown, the wire mesh 140 may be attached to the catheter body 110 and may encircle the catheter body 110. As shown, the wire mesh 140 may have an elongate, tubular shape, with a proximal end 142 and a distal end 144. In some embodiments, as shown, the wire mesh 140 may have a stent configuration, including a plurality of flexible wires that are woven or otherwise interconnected to form an expandable stent configuration. In some embodiments, the wire mesh 140 may be formed of a metal, such as cobalt-chromium or platinum-chromium. In some embodiments, the wire mesh 140 may be formed of a shape memory material, such as Nitinol. Still other suitable materials may be used for the wire mesh 140 in other embodiments. In some embodiments, the wire mesh 140 may be fixedly attached to the catheter body 110. In some embodiments, the distal end 144 of the wire mesh 140 may be fixedly attached to the catheter body 110 by the sleeve 160. For example, the sleeve 160 may be fixedly attached to the distal end 144 of the wire mesh 140 and to the catheter body 110. In some embodiments, the proximal end 142 of the wire mesh 140 may be removably attached to the catheter body 110. For example, the proximal end 142 of the wire mesh 140 may be removably attached to the catheter body 110 by an adhesive or other means of temporary attachment configured for separating the proximal end 142 of the wire mesh 140 from the catheter body 110 upon application of sufficient force. In some embodiments, the proximal end 142 of the wire mesh 140 may be unattached with respect to the catheter body 110. As shown, the wire mesh 140 may extend over the cuff 130. In some embodiments, the wire mesh 140 may be arranged such that the cuff 130 is positioned approximately midway between the proximal end 142 and the distal end 144 of the wire mesh 140, although other arrangements may be used.

The wire mesh 140 may be configured for radially expanding away from the catheter body 110 and the cuff 130. In particular, the wire mesh 140 may be configured for radially expanding from a radially collapsed configuration, as shown in FIGS. 2A-2D, and a radially expanded configuration, as shown in FIG. 2E. In this manner, when removal of the catheter 100 is desired, expansion of the wire mesh 140 may separate fibrous tissue growth from the cuff 130. As noted above, the distal end 144 of the wire mesh 140 may be fixedly attached to the catheter body 110, while the proximal end 142 of the wire mesh 140 maybe removably attached to the catheter body 110 or unattached to the catheter body 110. In some embodiments, the proximal end 142 of the wire mesh 140 maybe removably attached to the catheter body 110 by temporary means of attachment in order to inhibit the wire mesh 140 from inadvertently expanding while the catheter 100 is implanted in a patient. The wire mesh 140 may be configured for radially expanding away from the catheter body 110 and the cuff 130 by advancing the proximal end 142 of the wire mesh 140 toward the distal end 144 of the wire mesh 140. In other words, a force may be applied to the proximal end 142 of the wire mesh 140 to advance the proximal end 142 toward the distal end 144 of the wire mesh 140. In embodiments in which the proximal end 142 of the wire mesh 140 is removably attached to the catheter body 110, such force may be sufficient to break the temporary means of attachment, separating the proximal end 142 of the wire mesh 140 from the catheter body 110.

As shown, the collar 150 may be attached to the wire mesh 140 and may encircle the catheter body 110. In some embodiments, as shown, the collar 150 may be fixedly attached to the proximal end 142 of the wire mesh 140 and configured for being advanced therewith along the catheter body 110 toward the distal end 144 of the wire mesh 140. In some embodiments, the collar 150 may be removably attached to the catheter body 110. For example, the collar 150 may be removably attached to the catheter body 110 by an adhesive or other means of temporary attachment configured for separating the collar 150 from the catheter body 110 upon application of sufficient force. The wire mesh 140 may be configured for radially expanding away from the catheter body 110 and the cuff 130 by advancing the collar 150 (and the proximal end 142 of the wire mesh 140 attached thereto) toward the distal end 144 of the wire mesh 140. In other words, a force may be applied to the collar 150 to advance the proximal end 142 of the wire mesh 140 toward the distal end 144 of the wire mesh 140. In embodiments in which the collar 150 is removably attached to the catheter body 110, such force may be sufficient to break the temporary means of attachment, separating the collar 150 from the catheter body 110. In some embodiments, the collar 150 may be formed of a polymer, although other suitable materials may be used for the collar 150 in other embodiments. In some embodiments, as shown, the collar 150 may include a plurality of protrusions 152 configured for engaging mating features of the tool 200, as described below.

As shown in FIGS. 2C-2E, the tool 200 may be configured for positioning over a portion of the catheter body 110 and for removably coupling to the collar 150 to facilitate radial expansion of the wire mesh 140. The tool 200 may have an elongate, generally tubular shape, with a proximal end 202 and a distal end 204. In some embodiments, the tool 200 may include a first portion 210 and a second portion 214 pivotally coupled to one another by one or more hinges 214. In this manner, the tool 200 may have a clamshell configuration, with the first portion 210 and the second portion 212 being configured for pivoting between an open configuration for positioning the tool 200 relative to the portion of the catheter body 110 and a closed configuration for encircling the portion of the catheter body 110. In some embodiments, the tool 200 may include one or more locking mechanism configured for selectively retaining the first portion 210 and the second portion 212 in the closed configuration. In some embodiments, the tool 200 may be formed of a polymer, although other suitable materials may be used for the tool 200 in other embodiments. In some embodiments, the tool 200 may be configured for removably coupling to the collar 150 via a twist-lock mechanism. For example, as shown, the tool 200 may include a plurality of slots 126 configured for receiving the protrusions 152 to removably couple the tool 200 to the collar 150. As shown, one of the slots 216 may be formed in the first portion 210, and another of the slots 216 may be formed in the second portion 212. In some embodiments, as shown, each of the slots 126 may extend from the distal end 204 of the tool 200. In some embodiments, as shown, each of the slots 126 may have a contoured shape, such as an L shape or J shape, to provide a twist-lock mechanism with the protrusions 152. Other types of mechanisms and mating features may be used for removably coupling the tool 200 to the collar 150 in other embodiments. When the tool 200 is coupled to the collar 150, the tool 200 may be used to apply sufficient force to move the collar 150 and the proximal end 142 of the wire mesh 140 along the catheter body 110 to radially expand the wire mesh 140. In some embodiments, as shown, the tool 200 may include a plurality of wings 218 extending outward from the first portion 210 and the second portion 212 and configured for allowing a user to grasp and advance the tool 200.

The catheter 100 may be inserted into a patient's body using the current insertion procedure guidelines. When the catheter 100 is implanted, the cuff 130 may be positioned subcutaneously, while the collar 150 may remain outside of the patient's body. When desired, the catheter 100 may be easily removed from the patient, without need for significant dissection as is required with conventional tunneled catheters. In particular, the above-mentioned challenges in catheter removal may be addressed by the wire mesh 140 that surrounds the cuff 130 and the catheter body 110 and can be radially expanded with the help of the actuation tool 200. First, the tool 200 may be positioned around a portion of the catheter body 110 between the collar 150 and the coupler 122. In particular, the tool 200 may be positioned relative to the catheter body 110 while the first portion 210 and the second portion 212 are in the open configuration, and then the first portion 210 and the second portion 212 may be transitioned to the closed position to encircle the catheter body 110. Next, the tool 200 may be coupled to the collar 150 via the slots 216 of the tool 200 and the protrusions 152 of the collar 150. Then, the user may distally advance the tool 200 along the catheter body 110, thereby advancing the proximal end 142 of the wire mesh 140 toward the distal end 144 of the wire mesh 140 and thus radially expanding the wire mesh 140 away from the catheter body 110 and the cuff 130. In this manner, the tool 200 can be used to actuate the wire mesh 140, dissecting away the adhered tissue using compressive, tensile, and torsional force. In particular, expansion of the wire mesh 140 may dissect through the fibrous tissue adhered to the cuff 130, allowing for a safe and efficient removal procedure. Once the fibrous tissue is adequately disturbed, the catheter 100 can be removed from the patient by pulling the catheter 100 out.

The tool 200 and its interaction with the catheter 100 can be compared to an existing catheter extraction device, the OuTake Catheter Extractor (Merit Medical). The OuTake device is a simple design that attaches to a current clinical catheter and relies on user force to push and separate tissue from the cuff. The OuTake device has been observed to be bulky and ineffective, which may cause clinicians to revert back to using a blunt hemostat to remove the fibrous tissue. The design of the tool 200 and the catheter 100 described herein aims to eliminate the element of bulkiness to provide the physician and the patient with the most comfortable procedure possible. In use, the tool 200 may remain outside of the patient, and the wire mesh 140 covering the cuff 130 may be thin enough to not cause discomfort during prolonged implantation. As described above, the current procedure for catheter removal also requires an extensively trained physician to perform due to its nuanced nature. In contrast, with the wire mesh 140 and the extractor tool 200, the procedure will be more intuitive, and more providers may be able to perform the removal. In the future, this may include nurses and ER physicians who may need to remove a catheter during an emergency procedure. Ultimately, the improvements provided by the disclosed catheter 100 and tool 200 may increase the efficiency of the removal process.

A commercially available version of the disclosed system may include an injection-molded actuation tool 200, and a traditionally manufactured tunneled catheter 100 with an injection-molded connection point embedded into the catheter body 110, molded around a custom-braided wire mesh 140. Initial prototypes have been developed using a Hickman intravenous catheter, a 10 mm diameter nitinol biliary stent that has undergone a shape-setting heat treatment to reduce the diameter to 5 mm and meet the required characteristics, and a 3D printed plastic actuation tool and collar having protrusions. The initial prototypes have successfully demonstrated the desired interaction between the actuation tool, the collar, and the wire mesh.

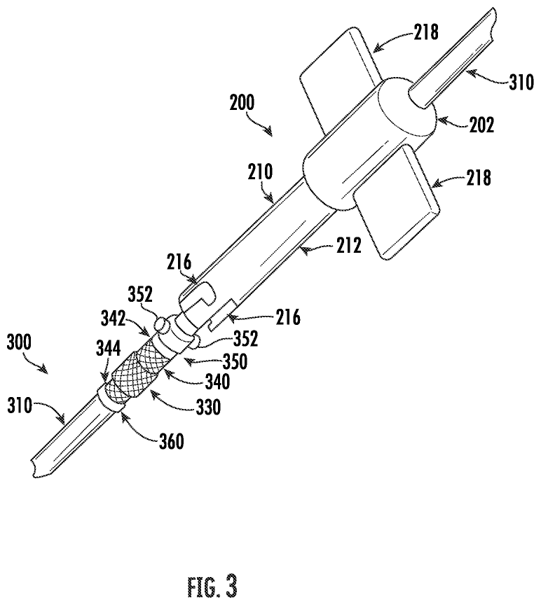

FIG. 3 illustrates another example catheter 300 in accordance with embodiments of the disclosure. Certain similarities and differences between the catheter 300 and the catheter 100 described above will be appreciated from the drawings and the following description. Corresponding features are identified using corresponding reference numbers. The catheter 300 similarly may be used with the tool 200 as a catheter system. As shown, the catheter may include a catheter body 310, a cuff 330, a wire mesh 340, a collar 350, and a sleeve 360. As described above, the cuff 330 may be configured for facilitating fibrous tissue growth upon implantation of the catheter 300 for a period of time, and the fibrous tissue may complicate removal of the catheter 300 from the patient. However, as described herein, the wire mesh 340 may be used to facilitate separation of the fibrous tissue growth from the cuff 330 when removal is desired.

The catheter body 310 may have an elongate, tubular shape and may include one or more lumens extending from a proximal end to a distal end of the catheter body 310 for delivering fluid into and/or withdrawing fluid from a patient. In some embodiments, similar to the catheter 100 described above, the catheter 300 may include multiple branches coupled to the catheter body 310 via a coupler, with each branch having an elongate, tubular shape including one or more lumens in fluid communication with one or more of the lumens of the catheter body 310.

The cuff 330 may be attached to the catheter body 310 and may encircle the catheter body 310, similar to a conventional cuff In some embodiments, the cuff 330 may be fixedly attached to the catheter body 310. The cuff 330 may be configured for facilitating fibrous tissue growth. In some embodiments, the cuff 330 may be formed of a knitted or woven material to facilitate tissue ingrowth. In some embodiments, the cuff 330 may be formed of PTE.

As shown, the wire mesh 340 may be attached to the catheter body 310 and may encircle the catheter body 310. As shown, the wire mesh 340 may have an elongate, tubular shape, with a proximal end 342 and a distal end 344. In some embodiments, as shown, the wire mesh 340 may have a stent configuration, including a plurality of flexible wires that are woven or otherwise interconnected to form an expandable stent configuration. In some embodiments, the wire mesh 340 may be formed of a metal, such as cobalt-chromium or platinum-chromium. In some embodiments, the wire mesh 340 may be formed of a shape memory material, such as Nitinol. Still other suitable materials may be used for the wire mesh 340 in other embodiments. In some embodiments, the wire mesh 340 may be fixedly attached to the catheter body 310. In some embodiments, the distal end 344 of the wire mesh 340 may be fixedly attached to the catheter body 310 by the sleeve 360. For example, the sleeve 360 may be fixedly attached to the distal end 344 of the wire mesh 340 and to the catheter body 310. In some embodiments, the proximal end 342 of the wire mesh 340 may be removably attached to the catheter body 310. For example, the proximal end 342 of the wire mesh 340 may be removably attached to the catheter body 310 by an adhesive or other means of temporary attachment configured for separating the proximal end 342 of the wire mesh 340 from the catheter body 310 upon application of sufficient force. In some embodiments, the proximal end 342 of the wire mesh 340 may be unattached with respect to the catheter body 310. As shown, the wire mesh 340 may extend over the cuff 330. In some embodiments, the wire mesh 340 may be arranged such that the cuff 330 is positioned approximately midway between the proximal end 342 and the distal end 344 of the wire mesh 340, although other arrangements may be used.

The wire mesh 340 may be configured for radially expanding away from the catheter body 310 and the cuff 330. In particular, the wire mesh 340 may be configured for radially expanding from a radially collapsed configuration, as shown in FIG. 3, and a radially expanded configuration. In this manner, when removal of the catheter 300 is desired, expansion of the wire mesh 340 may separate fibrous tissue growth from the cuff 330. As noted above, the distal end 344 of the wire mesh 340 may be fixedly attached to the catheter body 310, while the proximal end 342 of the wire mesh 340 maybe removably attached to the catheter body 310 or unattached to the catheter body 310. In some embodiments, the proximal end 342 of the wire mesh 340 may be removably attached to the catheter body 310 by temporary means of attachment in order to inhibit the wire mesh 340 from inadvertently expanding while the catheter 300 is implanted in a patient. The wire mesh 340 may be configured for radially expanding away from the catheter body 310 and the cuff 330 by advancing the proximal end 342 of the wire mesh 340 toward the distal end 344 of the wire mesh 340. In other words, a force may be applied to the proximal end 342 of the wire mesh 340 to advance the proximal end 342 toward the distal end 344 of the wire mesh 340. In embodiments in which the proximal end 342 of the wire mesh 340 is removably attached to the catheter body 310, such force may be sufficient to break the temporary means of attachment, separating the proximal end 342 of the wire mesh 340 from the catheter body 310.

As shown, the collar 350 may be attached to the wire mesh 340 and may encircle the catheter body 310. In some embodiments, as shown, the collar 350 may be fixedly attached to the proximal end 342 of the wire mesh 340 and configured for being advanced therewith along the catheter body 310 toward the distal end 344 of the wire mesh 340. In some embodiments, the collar 350 may be removably attached to the catheter body 310. For example, the collar 350 may be removably attached to the catheter body 310 by an adhesive or other means of temporary attachment configured for separating the collar 350 from the catheter body 310 upon application of sufficient force. The wire mesh 340 may be configured for radially expanding away from the catheter body 310 and the cuff 330 by advancing the collar 350 (and the proximal end 342 of the wire mesh 340 attached thereto) toward the distal end 344 of the wire mesh 340. In other words, a force may be applied to the collar 350 to advance the proximal end 342 of the wire mesh 340 toward the distal end 344 of the wire mesh 340. In embodiments in which the collar 350 is removably attached to the catheter body 310, such force may be sufficient to break the temporary means of attachment, separating the collar 350 from the catheter body 310. In some embodiments, the collar 350 may be formed of a polymer, although other suitable materials may be used for the collar 350 in other embodiments. In some embodiments, as shown, the collar 350 may include a plurality of protrusions 352 configured for engaging the slots 216 of the tool 200.

The catheter 300 may be implanted in a patient in a manner similar to the catheter 100 described above, with the cuff 330 being positioned subcutaneously, while the collar 350 remains outside of the patient's body. When desired, the catheter 300 may be easily removed from the patient using the tool 200, following the same steps as discussed above for removal of the catheter 100.

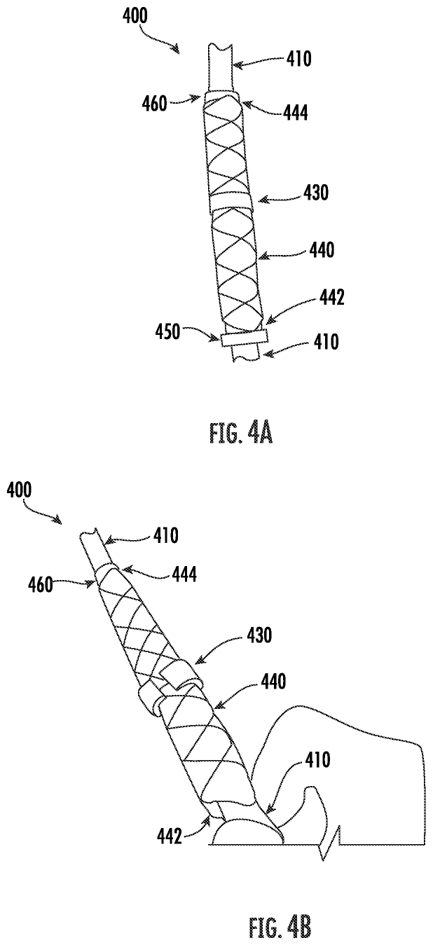

FIGS. 4A and 4B illustrate another example catheter 400 in accordance with embodiments of the disclosure. Certain similarities and differences between the catheter 400 and the catheters 100, 300 described above will be appreciated from the drawings and the following description. Corresponding features are identified using corresponding reference numbers. As shown, the catheter may include a catheter body 410, a cuff 430, a wire mesh 440, a temporary covering 450, and a sleeve 460. As described above, the cuff 430 may be configured for facilitating fibrous tissue growth upon implantation of the catheter 400 for a period of time, and the fibrous tissue may complicate removal of the catheter 400 from the patient. However, as described herein, the wire mesh 440 may be used to facilitate separation of the cuff 430 from the remainder of the catheter 400 when removal is desired.

The catheter body 410 may have an elongate, tubular shape and may include one or more lumens extending from a proximal end to a distal end of the catheter body 410 for delivering fluid into and/or withdrawing fluid from a patient. In some embodiments, similar to the catheter 100 described above, the catheter 400 may include multiple branches coupled to the catheter body 410 via a coupler, with each branch having an elongate, tubular shape including one or more lumens in fluid communication with one or more of the lumens of the catheter body 410.

As shown, the cuff 430 may encircle the catheter body 410 and the wire mesh 440, with the wire mesh 440 extending between the catheter body 410 and the cuff 430. In some embodiments, the cuff 430 may be removably attached to the wire mesh 440. For example, the cuff 430 may be removably attached to the wire mesh 440 by an adhesive or other means of temporary attachment configured for separating the cuff 430 from the wire mesh 440 upon application of sufficient force. The cuff 430 may be configured for facilitating fibrous tissue growth. In some embodiments, the cuff 430 may be formed of a knitted, woven, or porous material to facilitate tissue ingrowth. The cuff 430 may be formed of a biodegradable material, such that the cuff 430 may safely degrade within the patient's body after removal of the remainder of the catheter 400. The material of the cuff 430 may be selected to have a suitable degradation time in view of an intended period of implantation of the catheter 400. Conventional tunneled intravasular catheters are typically used for 6-8 months, with a typical survial rate of about one year. In some embodimeents, the material of the cuff 430 may be configured to degrade after 12 months in the patient's body. The material of the cuff 430 also may be selected to have a mechanical stength that is less than that of the wire mesh 440, such that the cuff 430 may be broken upon expansion of the wire mesh 440. In some embodiments, the cuff 430 may be formed of Poly (ϵ-caprolactone) (PCL), which has a degradtion time of about two years, can be processed as a porous scaffold and has a modulus of 0.4 GPa. In some embodiments, the cuff 430 may be formed of Poly (Lactic-co-Glycolic Acid) (PLGA), which has a tunable degradation time with six months being the low end degradation initiation similar to the lowest amount of time the catheter 400 typically may spend in the patient and a modulus of 2 GPa. In some embodiments, the cuff 430 may be formed of Poly (Lactic Acid) (PLA) or Poly (L-lactic Acid) (PLLA), the L-lactide specific version of PLA, with one year degradation timing being standard when these materials are used in polymeric stent applications. The modulus of PLA is slightly higher than that of PLGA at 3.5 GPa as it lacks glycolic acid, and PLLA is slightly less than PLA at 2.7 GPa. Still other suitable biocompatible and biodegradable materials may be used for the cuff 430 in other embodiments.

As shown, the wire mesh 440 may be attached to the catheter body 410 and may encircle the catheter body 410. As shown, the wire mesh 440 may have an elongate, tubular shape, with a proximal end 442 and a distal end 444. In some embodiments, as shown, the wire mesh 440 may have a stent configuration, including a plurality of flexible wires that are woven or otherwise interconnected to form an expandable stent configuration. In some embodiments, the wire mesh 440 may be formed of a metal, such as cobalt-chromium or platinum-chromium. In some embodiments, the wire mesh 440 may be formed of a shape memory material, such as Nitinol. Still other suitable materials may be used for the wire mesh 440 in other embodiments. In some embodiments, the wire mesh 440 may be fixedly attached to the catheter body 410. In some embodiments, the distal end 444 of the wire mesh 440 may be fixedly attached to the catheter body 410 by the sleeve 460. For example, the sleeve 460 may be fixedly attached to the distal end 444 of the wire mesh 440 and to the catheter body 410. In some embodiments, the proximal end 442 of the wire mesh 440 may be restrained relative to the catheter body 310 by the temporary covering 450. For example, as shown, the temporary covering 450 may extend over the proximal end 442 of the wire mesh 440 to inhibit movement of the proximal end 442 relative to the catheter body 410, but the removable covering 450 may be removed therefrom prior to removing the catheter 400 from the patient, as discussed below. As shown, the wire mesh 440 may extend between the catheter body 410 and the cuff 430. In some embodiments, the wire mesh 440 may be arranged such that the cuff 430 is positioned approximately midway between the proximal end 442 and the distal end 444 of the wire mesh 440, although other arrangements may be used.

The wire mesh 440 may be configured for radially expanding away from the catheter body 410. In particular, the wire mesh 440 may be configured for radially expanding from a radially collapsed configuration, as shown in FIG. 4A, and a radially expanded configuration, as shown in FIG. 4B. In this manner, when removal of the catheter 400 is desired, expansion of the wire mesh 440 may break the cuff 430 and separate the cuff 430 and the fibrous tissue adhered thereto from the remainder of the catheter 400. As noted above, the distal end 444 of the wire mesh 440 may be fixedly attached to the catheter body 410, while the proximal end 442 of the wire mesh 440 may be restrained relative to the catheter body 410 by the temporary covering 450, which may inhibit the wire mesh 440 from inadvertently expanding while the catheter 400 is implanted in a patient. The wire mesh 440 may be configured for radially expanding away from the catheter body 410 and breaking the cuff 430 by advancing the proximal end 442 of the wire mesh 440 toward the distal end 444 of the wire mesh 440. In other words, a force may be applied to the proximal end 442 of the wire mesh 440 to advance the proximal end 442 toward the distal end 444 of the wire mesh 440. For example, as shown in FIG. 4B, a user's fingers may press on the proximal end 442 of the wire mesh 440 to advance the proximal end 442 toward the distal end 444 of the wire mesh 440, thereby expanding the wire mesh 440 and breaking the cuff 430.

The catheter 400 may be implanted in a patient in a manner similar to the catheter 100 described above, with the cuff 430 being positioned subcutaneously, while the proximal end 442 of the wire mesh 440 remains outside of the patient's body. When desired, a user may advance the proximal end 442 of the wire mesh 440 toward the distal end 444 of the wire mesh 440, thereby expanding the wire mesh 440. This expansion may cause the cuff 430 to break and separate from the remainder of the catheter 400, allowing the remainder of the catheter 400 to be removed from the patient while the cuff 430 remains in the patient and safely degrades over time.

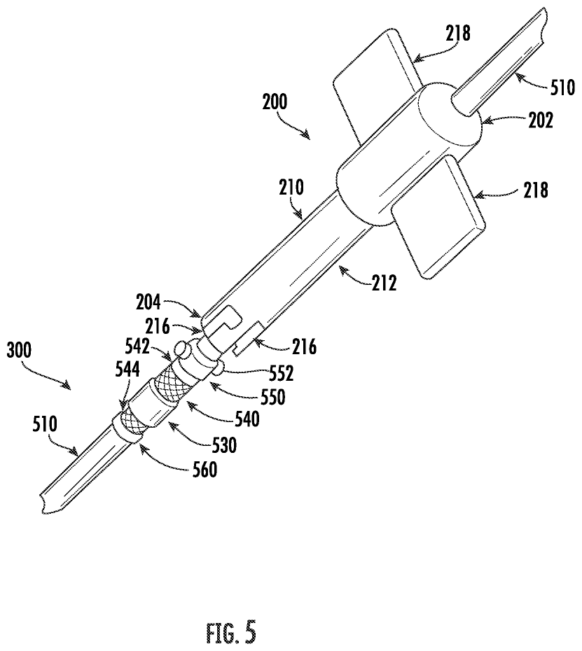

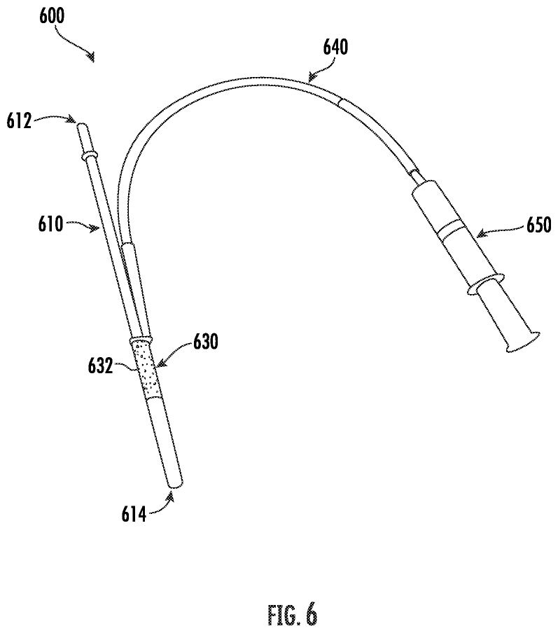

FIG. 5 illustrates another example catheter 500 in accordance with embodiments of the disclosure. Certain similarities and differences between the catheter 500 and the catheters 100, 300, 400 described above will be appreciated from the drawings and the following description. Corresponding features are identified using corresponding reference numbers. The catheter 500 similarly may be used with the tool 200 as a catheter system. As shown, the catheter may include a catheter body 510, a cuff 530, a wire mesh 540, a collar 550, and a sleeve 560. As described above, the cuff 530 may be configured for facilitating fibrous tissue growth upon implantation of the catheter 500 for a period of time, and the fibrous tissue may complicate removal of the catheter 500 from the patient. However, as described herein, the wire mesh 540 may be used to facilitate separation of the cuff 530 from the remainder of the catheter 500 when removal is desired.

The catheter body 510 may have an elongate, tubular shape and may include one or more lumens extending from a proximal end to a distal end of the catheter body 510 for delivering fluid into and/or withdrawing fluid from a patient. In some embodiments, similar to the catheter 100 described above, the catheter 500 may include multiple branches coupled to the catheter body 510 via a coupler, with each branch having an elongate, tubular shape including one or more lumens in fluid communication with one or more of the lumens of the catheter body 510.

As shown, the cuff 530 may encircle the catheter body 510 and the wire mesh 540, with the wire mesh 540 extending between the catheter body 510 and the cuff 530. In some embodiments, the cuff 530 may be removably attached to the wire mesh 540. For example, the cuff 530 may be removably attached to the wire mesh 540 by an adhesive or other means of temporary attachment configured for separating the cuff 530 from the wire mesh 540 upon application of sufficient force. The cuff 530 may be configured for facilitating fibrous tissue growth. In some embodiments, the cuff 530 may be formed of a knitted, woven, or porous material to facilitate tissue ingrowth. The cuff 530 may be formed of a biodegradable material, such that the cuff 530 may safely degrade within the patient's body after removal of the remainder of the catheter 500. The material of the cuff 530 may be selected to have a suitable degradation time in view of an intended period of implantation of the catheter 500. Conventional tunneled intravasular catheters are typically used for 6-8 months, with a typical survial rate of about one year. In some embodimeents, the material of the cuff 530 may be configured to degrade after 12 months in the patient's body. The material of the cuff 530 also may be selected to have a mechanical stength that is less than that of the wire mesh 540, such that the cuff 530 may be broken upon expansion of the wire mesh 540. In some embodiments, the cuff 530 may be formed of Poly (ϵ-caprolactone) (PCL), which has a degradtion time of about two years, can be processed as a porous scaffold and has a modulus of 0.4 GPa. In some embodiments, the cuff 530 may be formed of Poly (Lactic-co-Glycolic Acid) (PLGA), which has a tunable degradation time with six months being the low end degradation initiation similar to the lowest amount of time the catheter 500 typically may spend in the patient and a modulus of 2 GPa. In some embodiments, the cuff 530 may be formed of Poly (Lactic Acid) (PLA) or Poly (L-lactic Acid) (PLLA), the L-lactide specific version of PLA, with one year degradation timing being standard when these materials are used in polymeric stent applications. The modulus of PLA is slightly higher than that of PLGA at 3.5 GPa as it lacks glycolic acid, and PLLA is slightly less than PLA at 2.7 GPa. Still other suitable biocompatible and biodegradable materials may be used for the cuff 530 in other embodiments.

As shown, the wire mesh 540 may be attached to the catheter body 510 and may encircle the catheter body 510. As shown, the wire mesh 540 may have an elongate, tubular shape, with a proximal end 542 and a distal end 544. In some embodiments, as shown, the wire mesh 540 may have a stent configuration, including a plurality of flexible wires that are woven or otherwise interconnected to form an expandable stent configuration. In some embodiments, the wire mesh 540 may be formed of a metal, such as cobalt-chromium or platinum-chromium. In some embodiments, the wire mesh 540 may be formed of a shape memory material, such as Nitinol. Still other suitable materials may be used for the wire mesh 540 in other embodiments. In some embodiments, the wire mesh 540 may be fixedly attached to the catheter body 510. In some embodiments, the distal end 544 of the wire mesh 540 may be fixedly attached to the catheter body 510 by the sleeve 560. For example, the sleeve 560 may be fixedly attached to the distal end 544 of the wire mesh 540 and to the catheter body 510. In some embodiments, the proximal end 542 of the wire mesh 540 may be removably attached to the catheter body 510. For example, the proximal end 542 of the wire mesh 540 may be removably attached to the catheter body 510 by an adhesive or other means of temporary attachment configured for separating the proximal end 542 of the wire mesh 540 from the catheter body 510 upon application of sufficient force. In some embodiments, the proximal end 542 of the wire mesh 540 may be unattached with respect to the catheter body 510. As shown, the wire mesh 540 may extend between the catheter body 510 and the cuff 530. In some embodiments, the wire mesh 540 may be arranged such that the cuff 530 is positioned approximately midway between the proximal end 542 and the distal end 544 of the wire mesh 540, although other arrangements may be used.

The wire mesh 540 may be configured for radially expanding away from the catheter body 510. In particular, the wire mesh 540 may be configured for radially expanding from a radially collapsed configuration, as shown in FIG. 5, and a radially expanded configuration. In this manner, when removal of the catheter 500 is desired, expansion of the wire mesh 540 may break the cuff 530 and separate the cuff 530 and the fibrous tissue adhered thereto from the remainder of the catheter 500. As noted above, the distal end 544 of the wire mesh 540 may be fixedly attached to the catheter body 510, while the proximal end 542 of the wire mesh 540 may be removably attached to the catheter body 510 or unattached to the catheter body 510. In some embodiments, the proximal end 542 of the wire mesh 540 maybe removably attached to the catheter body 510 by temporary means of attachment in order to inhibit the wire mesh 540 from inadvertently expanding while the catheter 500 is implanted in a patient. The wire mesh 540 may be configured for radially expanding away from the catheter body 510 and breaking the cuff 530 by advancing the proximal end 542 of the wire mesh 540 toward the distal end 544 of the wire mesh 540. In other words, a force may be applied to the proximal end 542 of the wire mesh 540 to advance the proximal end 542 toward the distal end 544 of the wire mesh 540. In embodiments in which the proximal end 542 of the wire mesh 540 is removably attached to the catheter body 510, such force may be sufficient to break the temporary means of attachment, separating the proximal end 542 of the wire mesh 540 from the catheter body 510.

As shown, the collar 550 may be attached to the wire mesh 540 and may encircle the catheter body 510. In some embodiments, as shown, the collar 550 may be fixedly attached to the proximal end 542 of the wire mesh 540 and configured for being advanced therewith along the catheter body 510 toward the distal end 544 of the wire mesh 540. In some embodiments, the collar 550 may be removably attached to the catheter body 510. For example, the collar 550 may be removably attached to the catheter body 510 by an adhesive or other means of temporary attachment configured for separating the collar 550 from the catheter body 510 upon application of sufficient force. The wire mesh 540 may be configured for radially expanding away from the catheter body 510 and breaking the cuff 530 by advancing the collar 550 (and the proximal end 542 of the wire mesh 540 attached thereto) toward the distal end 544 of the wire mesh 540. In other words, a force may be applied to the collar 550 to advance the proximal end 542 of the wire mesh 540 toward the distal end 544 of the wire mesh 540. In embodiments in which the collar 550 is removably attached to the catheter body 510, such force may be sufficient to break the temporary means of attachment, separating the collar 550 from the catheter body 510. In some embodiments, the collar 550 may be formed of a polymer, although other suitable materials may be used for the collar 550 in other embodiments. In some embodiments, as shown, the collar 550 may include a plurality of protrusions 552 configured for engaging the slots 216 of the tool 200.

The catheter 500 may be implanted in a patient in a manner similar to the catheter 100 described above, with the cuff 530 being positioned subcutaneously, while the collar 550 remains outside of the patient's body. When desired, the catheter 500 may be easily removed from the patient using the tool 200 in a manner similar to that described above. In particular, a user may couple the tool 200 to the collar 550 and advance the collar 550 along the catheter body 510 toward the distal end 544 of the wire mesh 540, thereby expanding the wire mesh 540. This expansion may cause the cuff 530 to break and separate from the remainder of the catheter 500, allowing the remainder of the catheter 500 to be removed from the patient while the cuff 530 remains in the patient and safely degrades over time.

FIG. 6 illustrates another example catheter 600 in accordance with embodiments of the disclosure. Certain similarities and differences between the catheter 600 and the catheters 100, 300, 400, 500 described above will be appreciated from the drawings and the following description. Corresponding features are identified using corresponding reference numbers. The catheter 600 may be used with an inflation device 650 as a catheter system. As shown, the catheter may include a catheter body 610, a cuff 630, a balloon 632, and a tube 640. As described above, the cuff 630 may be configured for facilitating fibrous tissue growth upon implantation of the catheter 600 for a period of time, and the fibrous tissue may complicate removal of the catheter 600 from the patient. However, as described herein, the balloon 632 may be used to facilitate separation of the fibrous tissue growth from the cuff 630 when removal is desired.

The catheter body 610 may have an elongate, tubular shape and may include one or more lumens extending from a proximal end to a distal end of the catheter body 610 for delivering fluid into and/or withdrawing fluid from a patient. In some embodiments, similar to the catheter 100 described above, the catheter 600 may include multiple branches coupled to the catheter body 610 via a coupler, with each branch having an elongate, tubular shape including one or more lumens in fluid communication with one or more of the lumens of the catheter body 610.

The cuff 630 may encircle the catheter body 610 and the balloon 632, with the balloon 632 being positioned between the catheter body 610 and the cuff 630. The cuff 630 may be fixedly attached to the balloon 632. The cuff 630 may be configured for facilitating fibrous tissue growth. In some embodiments, the cuff 630 may be a coating that is fixedly attached to the balloon 632. For example, the cuff 630 may be a coating including heparin or another porous binding agent configured for inducing fibrin attachment thereto. Various other suitable materials and configurations of the cuff 630 may be used in other embodiments.

The balloon 632 may be fixedly attached to the catheter body 610 and may encircle the catheter body 610. The balloon 632 may be configured in a manner similar to an angioplasty balloon. The balloon 632 may be configured for radially expanding away from the catheter body 610. In particular, the balloon 632 may be configured for radially expanding from a radially collapsed configuration, as shown in FIG. 6, to a radially expanded configuration. In this manner, when removal of the catheter 600 is desired, expansion of the balloon 632 may cause the fibrous tissue adhered to the cuff 630 to separate from the cuff 630. The tube 640 may be coupled to the balloon 632 and may include a lumen that is in fluid communication with an internal reservoir of the balloon 632. In this manner, a fluid may be passed through the tube 640 and into the internal reservoir to expand the balloon 632, and the fluid may be withdrawn through the tube 640 to allow the balloon 632 to collapse. As shown, the inflation device 650 may be removably coupled to the free end of the tube 640 for introducing and withdrawing the fluid. In some embodiments, as shown, the inflation device 650 may be a syringe, although other configurations of the inflation device 650 may be used in other embodiments.

The catheter 600 may be implanted in a patient in a manner similar to the catheter 100 described above, with the cuff 630 being positioned subcutaneously, while the tube 640 extends outside of the patient's body. When desired, the catheter 600 may be easily removed from the patient using the inflation device 650. In particular, a user may couple the inflation device 650 to the tube 640 and deliver a fluid into the internal reservoir of the balloon 632, thereby expanding the balloon 632. This expansion may cause the fibrous tissue adhered to the cuff 630 to separate from the cuff 630, allowing the catheter 600 to be removed from the patient.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment. The term “based at least in part on” and “based on” are synonymous terms which may be used interchangeably herein.

What is claimed is:

1. A catheter comprising:
a catheter body having a tubular shape;
a cuff encircling the catheter body, wherein the cuff is configured for facilitating fibrous tissue growth; and
a wire mesh attached to and encircling the catheter body, wherein the wire mesh is configured for radially expanding away from the catheter body.

2. The catheter of claim 1, wherein the catheter is a tunneled catheter.

3. The catheter of claim 1, wherein the cuff is formed of a knitted or woven material.

4. The catheter of claim 1, wherein the cuff is formed of polyethylene terephthalate.

5. The catheter of claim 1, wherein the wire mesh has a stent configuration.

6. The catheter of claim 1, wherein the wire mesh extends over the cuff and is configured for radially expanding away from the cuff.

7. The catheter of claim 6, wherein the cuff is fixedly attached to the catheter body.

8. The catheter of claim 1, wherein the wire mesh extends between the catheter body and the cuff and is configured for breaking the cuff upon radial expansion of the wire.

9. The catheter of claim 8, wherein the cuff is removably attached to the wire mesh.

10. The catheter of claim 1, wherein the wire mesh has a distal end and a proximal end, and wherein the distal end of the wire mesh is fixedly attached to the catheter body.

11. The catheter of claim 10, wherein the wire mesh is configured for radially expanding away from the catheter body by advancing the proximal end of the wire mesh toward the distal end of the wire mesh.

12. The catheter of claim 10, wherein the proximal end of the wire mesh is removably attached to the catheter body.

13. The catheter of claim 1, further comprising a collar fixedly attached to the wire mesh and encircling the catheter body.

14. The catheter of claim 13, wherein the collar is removably attached to the catheter body.

15. A catheter system comprising:
a catheter comprising:
a catheter body having a tubular shape;
a cuff attached to and encircling the catheter body, wherein the cuff is configured for facilitating fibrous tissue growth;
a wire mesh attached to and encircling the catheter body, wherein the wire mesh extends over the cuff and is configured for radially expanding away from the cuff; and
a collar attached to the wire mesh and encircling the catheter body; and
a tool configured for positioning over a portion of the catheter body and for removably coupling to the collar to facilitate radial expansion of the wire mesh.

16. The catheter system of claim 15, wherein the tool is configured for removably coupling to the collar via a twist-lock mechanism.

17. The catheter system of claim 15, wherein the collar comprises a plurality of protrusions, and wherein the tool comprises a plurality of slots configured for receiving the protrusions to removably couple the tool to the collar.

18. A method for removing a catheter from a patient, the catheter including a catheter body, a cuff with fibrous tissue growth, and a wire mesh encircling the catheter body, the method comprising:
radially expanding the wire mesh away from the catheter body; and
pulling at least the catheter body and the wire mesh out of the patient.

19. The method of claim 18, wherein the wire mesh extends over the cuff, wherein radially expanding the wire mesh away from the catheter body comprises radially expanding the wire mesh away from the cuff and separating the fibrous tissue growth from the cuff, and wherein pulling at least the catheter body and the wire mesh out of the patient comprises pulling the catheter body, the wire mesh, and the cuff out of the patient.

20. The method of claim 19, wherein the wire mesh extends between the catheter body and the cuff, and wherein radially expanding the wire mesh away from the catheter body comprises breaking the cuff and separating the wire mesh from the cuff.
